(19) United States
(12) Patent Application Publication
(10) Pub. No.: US 2018/0214131 A1
(43) Pub. Date: Aug. 2, 2018

(54) METHOD AND SYSTEM FOR UPLOADING ULTRASONIC DIAGNOSIS IMAGE

(71) Applicant: HEALCERION CO., LTD., Seoul (KR)

(72) Inventors: Jeong Won RYU, Seoul (KR); You Chan CHOUNG, Seoul (KR)

(21) Appl. No.: 15/747,941

(22) PCT Filed: Aug. 16, 2016
(86) PCT No.: PCT/KR2016/008969

§ 371 Date: Jan. 26, 2018

(30) Foreign Application Priority Data
Aug. 18, 2015 (KR) 10-2015-0116135

(51) Int. Cl. A61B 8/00; G16H 30/40

(52) U.S. Cl. CPC A61B 8/468; H04L 67/2823; G16H 30/40; A61B 8/463

(57) ABSTRACT

Disclosed are a method and a system for uploading an ultrasonic diagnosis image. The method may include generating an ultrasonic image to be uploaded, by extracting an ultrasonic image part from an ultrasonic diagnosis image, generating a hash tag by converting at least a part of ultrasonic diagnosis image information corresponding to the ultrasonic diagnosis image, into text data, and uploading the ultrasonic image to be uploaded and the hash tag together on the Internet service.

### CROSS-REFERENCE TO RELATED APPLICATION

This application claims priority to and the benefit of Korean Patent Application No. 10-2015-0116135, filed on Aug. 18, 2015, the disclosure of which is incorporated herein by reference in its entirety.

### FIELD

The present invention relates to a method and a system for uploading an ultrasonic diagnosis image, and more particularly, to a method and a system for converting ultrasonic diagnosis information corresponding to an ultrasonic diagnosis image into a hash tag and uploading them on the Internet service while uploading an ultrasonic image part of the ultrasonic diagnosis image on the Internet service.

### BACKGROUND

Due to noninvasive and nondestructive properties thereof, an ultrasonic diagnosis is generally used in the medical field to obtain information of the inside of a target. Since it is possible to provide a high-resolution image of internal organizations of a subject to be inspected to a doctor without surgical operations of directly incising and observing the subject, an ultrasonic diagnosis is very importantly used in the medical field.

An ultrasonic diagnostic system is a system which emits an ultrasonic signal from a body surface of a subject toward a target area inside the body, extracts information from a reflected ultrasonic signal, and noninvasively obtains an image of a section of soft tissue or a blood flow.

In comparison to other diagnostic imaging apparatuses such as an X-ray inspection apparatus, a computerized tomography (CT) scanner, a magnetic resonance image (MRI) scanner, and a nuclear medicine inspection apparatus, due to a small size, a low price, a display ability in real time, and high safety without being exposed to X-rays and the like, such ultrasonic diagnostic systems described above are generally used to diagnose a heart, abdominal organs, a urinary system, genitals, and the like.

Recently, there have been attempts to embodying a portable ultrasonic diagnosis apparatus and connecting a portable terminal such as a smart phone and a tablet with the ultrasonic diagnosis apparatus through wireless communication to perform an ultrasonic diagnosis. The portable ultrasonic diagnosis apparatus, due to mobility thereof, is generally used across several doctor's offices or hospital rooms and is shared by several users.

An ultrasonic diagnosis image obtained as a result of performing an ultrasonic diagnosis by the portable ultrasonic diagnosis apparatus or a general ultrasonic diagnosis apparatus (an immobile ultrasonic diagnosis apparatus) includes source data of the ultrasonic diagnosis image, patient information, diagnosis environment information, diagnosis information, and the like. The source data of the ultrasonic diagnosis image shows only an ultrasonic image of a patient obtained by the ultrasonic diagnosis apparatus. The patient information shows information on a patient who is ultrasonic-diagnosed and includes a name, age, height, weight, gender, and the like. The diagnosis environment information and diagnosis information includes a scale of an image obtained by the ultrasonic diagnosis apparatus, myocardial infarction (MI), tumor in situ (TIS), frame per second (FPS), and the like.

The ultrasonic diagnosis image is shared on the Internet service such as social network service (SNS) and the like and is generally used as a reference material for diagnosing a patient's condition. That is, a large number of ultrasonic diagnosis images are shared on the Internet service and are generally used for education of medical students, joint treatment and study of medical personnel, and the like. Also, ultrasonic diagnosis images shared on the Internet service are helpful for emergency crews or medical doctors to recognize a patient's condition by comparing an ultrasonic diagnosis image obtained by diagnosing the patient using a portable ultrasonic diagnosis apparatus with the shared ultrasonic diagnosis images.

However, to upload an ultrasonic diagnosis image on the Internet, an image is extracted by performing a screenshot function or copying function on a monitor screen on which the ultrasonic diagnosis image is shown. Currently, since it is more necessary to upload a large number of ultrasonic diagnosis images with high resolution for education or joint treatment, there are many cases of giving up uploading images due to inconvenience of manual uploading during uploading.

Also, to allow ultrasonic diagnosis images to be properly used in cases of education, joint treatment, and emergent patients, it is necessary to upload the ultrasonic diagnosis image with pieces of information for determining which body part an uploaded ultrasonic diagnosis image is, in which condition the uploaded ultrasonic diagnosis image is captured, to which disease the uploaded ultrasonic diagnosis image relates, and the like. However, currently, since doctors and the like who upload an image have to manually combine these pieces of information with the image one by one using a tag format, it is very inconvenient. Also, since tag formats are different for each types of Internet service such as google, twitter, facebook, and the like, even when users search for a necessary ultrasonic diagnosis image through a tag, there is a difficulty in searching for a proper image.

Also, although it is necessary for medical students in ultrasonic practices to study by comparing a variety of precise ultrasonic diagnosis images, since a large number of ultrasonic diagnosis images are not collected due to an existing manual method of uploading an ultrasonic diagnosis image, it is difficult to properly perform ultrasonic practice education of medical students.

In addition, an ultrasonic diagnosis image includes personal information for identifying a patient who is alive, for example, a name. When the ultrasonic diagnosis image including the personal information is exposed on the Internet service such as SNS and the like due to negligence of a person who uploads the ultrasonic diagnosis image, a danger of exposing the personal information of the patient may occur. Since it may violate personal data protection act implemented in Korea from September 30, 2011, caution is necessary.

Also, when to search for an ultrasonic diagnosis image shared on the Internet service such as SNS and the like, it is necessary to refer to a document drafted with the shared ultrasonic diagnosis image. When content of the document is incomplete or there is no document, it is very difficult to search for an image.

### SUMMARY

It is an aspect of the present invention to provide a method and a system for uploading an ultrasonic diagnosis image, capable of automatically converting an ultrasonic diagnosis image into a format adequate for the Internet service and automatically converting information related to the ultrasonic diagnosis image into a hash tag format to upload the image with the hash tag together when to share the ultrasonic diagnosis image on the Internet service such as social network service (SNS) and the like.

It is another aspect of the present invention to provide a method and a system for uploading an ultrasonic diagnosis image to allow a user to easily search for an ultrasonic diagnosis image on the Internet service such as SNS and the like using a hash tag.

Aspects of the present invention are not limited to the above description and additional unmentioned aspects of the present invention will be understood by those skilled in the art from the following description.

According to one aspect of the present invention, a method of uploading an ultrasonic diagnosis image includes generating an ultrasonic image to be uploaded, by extracting an ultrasonic image part from an ultrasonic diagnosis image, generating a hash tag by converting at least a part of ultrasonic diagnosis image information corresponding to the ultrasonic diagnosis image, into text data, and uploading the ultrasonic image to be uploaded and the hash tag together on the Internet service.

The generating of the ultrasonic image to be uploaded may include, when the extracted ultrasonic image part is a video, generating an ultrasonic image to be uploaded, by compressing the extracted ultrasonic image part using a standard video coding method.

The generating of the ultrasonic image to be uploaded may include, when the extracted ultrasonic image part is a still image, generating an ultrasonic image to be uploaded, by compressing the extracted ultrasonic image part using a standard still image coding method or generating an ultrasonic image to be uploaded, which has a standard incompressible still image format.

The at least some pieces of information may include at least one of diagnosis environment information, patient information, and patient diagnosis information. The diagnosis environment information may include at least one of diagnosis equipment, a diagnosis position, and a diagnosis time. The patient information may include at least one of a gender, age, height, and weight. The patient diagnosis information may include at least one of a diagnosed part, a diagnosis field, and a diagnosis result.

According to another aspect of the present invention, an ultrasonic diagnosis image uploading system includes an ultrasonic diagnosis apparatus and an uploading terminal which includes an ultrasonic image generator which generates an ultrasonic image to be uploaded, by extracting an ultrasonic image part from an ultrasonic diagnosis image obtained through the ultrasonic diagnosis apparatus, a text converter which converts at least some pieces of ultrasonic diagnosis image information corresponding to the ultrasonic diagnosis image, into text data, a tag generator which generates a hash tag for each piece of information converted into the text data, and an uploader which uploads the ultrasonic image to be uploaded and the hash tag together on the Internet service.

According to still another aspect of the present invention, an ultrasonic diagnosis apparatus includes an ultrasonic probe which generates an ultrasonic diagnosis image signal by converting an ultrasonic signal reflected by a subject to be inspected, a diagnosis image generator which generates an ultrasonic diagnosis image using the ultrasonic diagnosis image signal obtained through the ultrasonic probe, an ultrasonic image generator which generates an ultrasonic image to be uploaded, by extracting an ultrasonic image part from the ultrasonic diagnosis image generated by the diagnosis image generator, a text converter which converts at least some pieces of ultrasonic diagnosis image information corresponding to the ultrasonic diagnosis image, into text data, a hash tag generator which generates a hash tag for each piece of information converted into the text data, and an uploader which uploads the ultrasonic image to be uploaded and the hash tag on the Internet service.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIGS. 1A and 1B are views illustrating components of an ultrasonic diagnosis image uploading system according to one embodiment of the present invention;

FIG. 2 is a configuration diagram of an uploading terminal according to one embodiment of the present invention;

FIG. 3 is a view illustrating and example of an ultrasonic diagnosis image user interface (UI) output on a display screen of the uploading terminal;

FIG. 4 is a view illustrating an example of an ultrasonic image part extracted from an ultrasonic diagnosis image;

FIG. 5 is a view illustrating a hash tag on pieces of information to be uploaded with an ultrasonic image to be uploaded on the Internet service;

FIG. 6 is a flowchart illustrating an ultrasonic diagnosis image uploading method according to one embodiment of the present invention; and

FIG. 7 is a flowchart illustrating a method of generating an ultrasonic image to be uploaded according to one embodiment of the present invention.

### DETAILED DESCRIPTION

Hereinafter, exemplary embodiments of the present invention will be described in detail with reference to the attached drawings.

The embodiments of the present invention are provided to more completely explain the present invention to one of ordinary skill in the art. The following embodiments may be modified into various different forms, and the scope of the present invention is not limited thereto. The embodiments are provided to make the disclosure more substantial and complete and to completely convey the concept to those skilled in the art.

The terms used herein are to explain particular embodiments but are not intended to limit the present invention. As used herein, singular forms, unless contextually defined otherwise, may include plural forms. Also, the terms “comprise” and/or “comprising” are used herein to specify the present of stated shapes, numbers, steps, operations, members, elements, and/or groups thereof but do not preclude the presence or addition of one or more other shapes, numbers, operations, members, elements and/or groups thereof. As used herein, the term “and/or” includes any and all combinations or one of a plurality of associated listed items.

It is obvious that although the terms “first”, “second”, and the like are used herein to describe various members, areas, layers, and/or portions, these members, areas, layers, and/or portions are not limited by these terms. These terms do not mean particular order, top and bottom, or ratings and are used only to distinguish one member, area, or portion from another member, area, or portion. Accordingly, a first member, area, or portion, which will be described below, may be referred to as a second member, area, or portion without departing from the scope of the present invention.

Hereinafter, the embodiments of the present invention will be described with reference to the drawings which schematically illustrate the embodiments. In the drawings, for example, depending on a manufacturing technology and/or tolerance, modifications of illustrated shapes may be perceived. Accordingly, it should be understood that the embodiments of the present invention are not limited to particular shapes in areas shown in the specification and may include, for example, changes in shape caused during a manufacturing process.

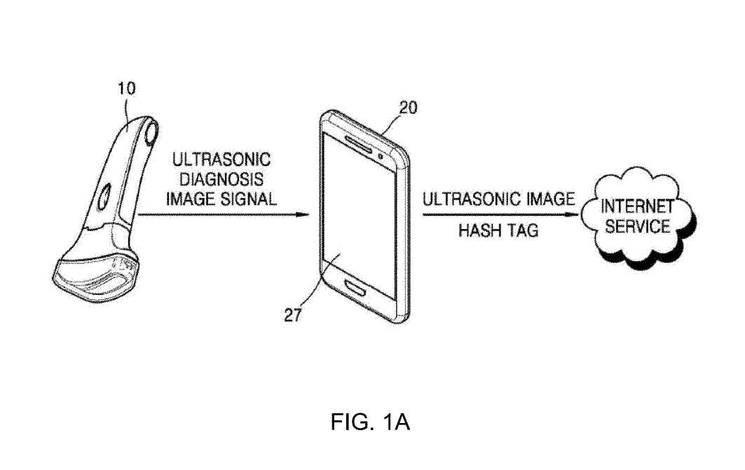

FIGS. 1A and 1B are views illustrating components of an ultrasonic diagnosis image uploading system according to one embodiment of the present invention.

FIG. 1A illustrates a portable ultrasonic diagnosis apparatus, and FIG. 1B illustrates an ultrasonic diagnosis apparatus installed in a clinic or the like.

Referring to FIG. 1A, the ultrasonic diagnosis image uploading system according to one embodiment of the present invention includes an ultrasonic diagnosis apparatus 10 and an uploading terminal 20.

The ultrasonic diagnosis apparatus 10 of FIG. 1A describes an ultrasonic probe which transmits an ultrasonic signal to a subject to be inspected and transmits frame data obtained from an echo signal reflected from the object. Although being shown as a wireless portable ultrasonic probe, the ultrasonic probe of FIG. 1A may be a wired type and may perform even a function of converting frame data into an ultrasonic diagnosis image signal and transmitting the signal to the uploading terminal 20.

Accordingly, the ultrasonic diagnosis image signal shown in FIG. 1A may refer to the frame data or may include the ultrasonic diagnosis image signal converted from the frame data.

The ultrasonic diagnosis apparatus 10 of FIG. 1A may include a beam former (not shown) which generates an ultrasonic signal and collects an echo signal to generate frame data, a signal generator (not shown) which generates an ultrasonic diagnosis image signal from the frame data, and a communication module (not shown) which transmits and receives data with the uploading terminal 20. Also, the ultrasonic diagnosis apparatus 10 may be manufactured to be portable or nonportable.

The uploading terminal 20 may be a mobile terminal or an immobile terminal and may include a terminal which has an operation system, is Internet-accessible, and allows a variety of applications to be installed therein, for example, a smart phone, a tablet personal computer (PC), a laptop PC, or an immovable PC.

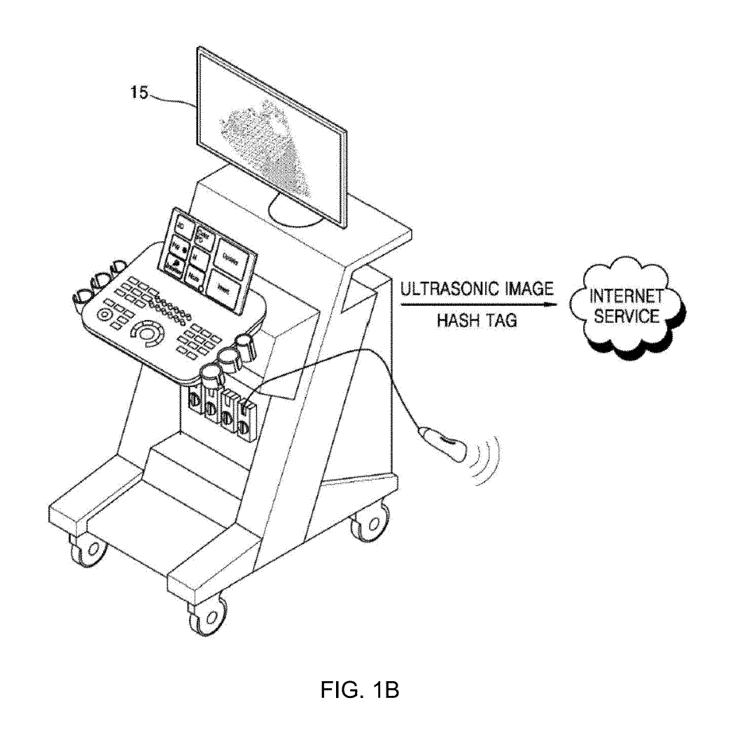

The uploading terminal 20, as shown in FIG. 2, includes an ultrasonic image generator 22, a text converter 23, a hash tag generator 24, and an uploader 26.

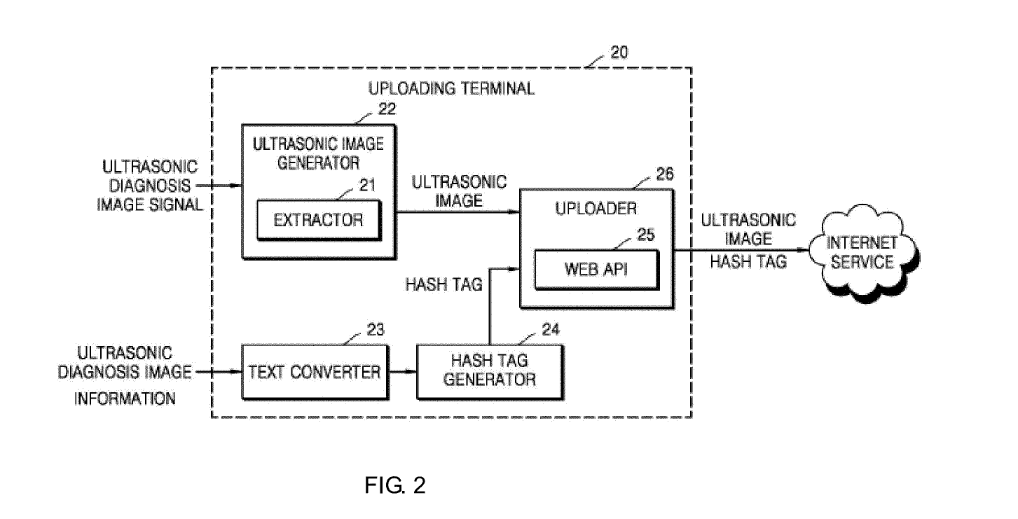

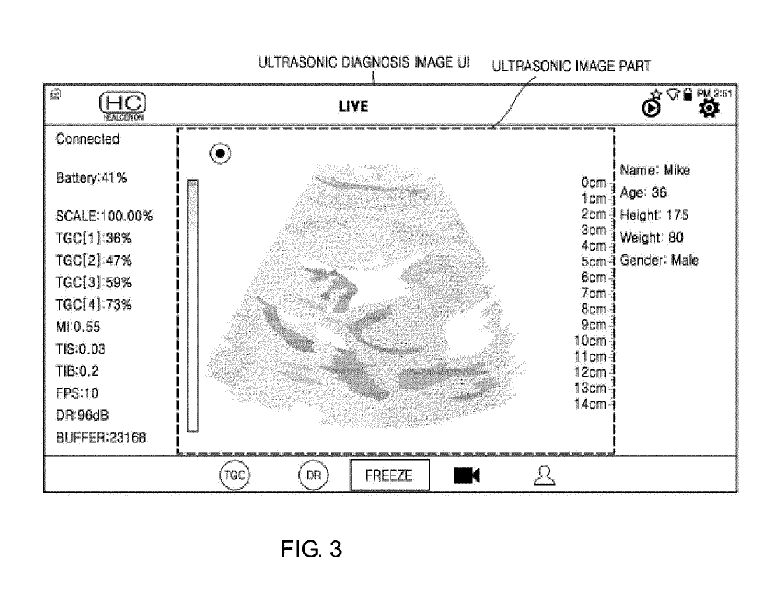

The ultrasonic image generator 22 includes an extractor 21 which extracts an ultrasonic image part from an ultrasonic diagnosis image obtained from an ultrasonic diagnosis image signal transmitted from the ultrasonic diagnosis apparatus 10 and generates an ultrasonic image to be uploaded, using the extracted ultrasonic image part extracted by the extractor 21. The ultrasonic diagnosis image, as shown in FIG. 3, may be provided to a user through an ultrasonic diagnosis image user interface (UI) output on a display screen 27 of the uploading terminal 20. The extractor 21, as shown in FIG. 4, extracts only the ultrasonic image part from the ultrasonic diagnosis image output through the ultrasonic diagnosis image UI.

The ultrasonic image generator 22 may generate an ultrasonic diagnosis image according to resolution of the display screen 27 of the uploading terminal 20 and may transmit the ultrasonic diagnosis image to the uploading terminal 20.

Here, the ultrasonic diagnosis image may include ultrasonic diagnosis image information in addition to the ultrasonic image part. Accordingly, the extractor 21 extracts the ultrasonic image part and the ultrasonic diagnosis image information from the ultrasonic diagnosis image. Meanwhile, the ultrasonic diagnosis image information may be not included in the ultrasonic diagnosis image. In this case, the user may directly input the ultrasonic diagnosis image information into the uploading terminal 20.

A process of generating, by the ultrasonic image generator 22, an ultrasonic image to be uploaded will be described.

A method of generating, by the ultrasonic image generator 22, an ultrasonic image to be uploaded may vary depending on whether an ultrasonic image part extracted by the extractor 21 is a video or a still image.

First, when the extracted ultrasonic image part is a video, the ultrasonic image generator 22 may generate an ultrasonic image to be uploaded, by compressing the ultrasonic image part which is a video, using a standard video coding method. Here, the standard video coding method may be Moving Picture Experts Group 4 (MPEG4) and the like.

Meanwhile, when the extracted ultrasonic image part is a still image, depending on whether the user selects compression, an ultrasonic image to be uploaded may be generated by compressing the ultrasonic image part which is a still image using a standard still image coding method or an ultrasonic image to be uploaded, which has a standard incompressible still image format, may be generated.

That is, when compression is selected, the ultrasonic image generator 22 generates an ultrasonic image to be uploaded, by compressing the ultrasonic image part which is a still image, using a standard still image coding method. Here, a format of the ultrasonic image to be uploaded, which is generated by being compressed using the standard still image coding method may be Joint Photographic coding Experts Group (JPEG) and the like. A selection of the user for compression may be performed using an input device (not shown) provided at the uploading terminal 20, for example, a compression selection key button and the like.

When compression is not selected, the ultrasonic image generator 22 generates an ultrasonic image to be uploaded, which has the standard incompressible still image format, using the ultrasonic image part which is a still image. Here, the standard incompressible still image format may be a Portable Network Graphics (PNG) and the like.

The text converter 23 converts at least some pieces of the ultrasonic diagnosis image information corresponding to the ultrasonic diagnosis image, into text data. Here, the ultrasonic diagnosis image information, as described above, may be provided by the extractor 21 or may be provided by the user directly inputting to the uploading terminal 20. Also, when information, to be transmitted to the hash tag generator 24, of the ultrasonic diagnosis image information already has a text data format, the text converter 23 may transmit the information to the hash tag generator 24 without conversion into text data.

The ultrasonic diagnosis image information may include diagnosis environment information in which an ultrasonic diagnosis is performed, personal identification information provided by personal data protection act implemented in Korea, patient information irrelevant to the personal identification information, and patient diagnosis information according to an ultrasonic diagnosis.

The diagnosis environment information may include information related to diagnosis equipment, a diagnosis position, and diagnosis time. The diagnosis equipment refers to the ultrasonic diagnosis apparatus 10, and the diagnosis position is a position at which an ultrasonic diagnosis is performed and which may be recognized through a direct input by a user or a global positioning system (GPS) provided at the ultrasonic diagnosis apparatus 10 or the uploading terminal 20. The diagnosis time is a time when an ultrasonic diagnosis is performed and may be measured by an internal timer of the ultrasonic diagnosis apparatus 10 or the uploading terminal 20 or may be input by a user.

The personal identification information is personal data provided by personal data protection act, that is, information which relates to a person, who is alive, allows the person to be identified through a name, a residence number, and the like corresponding to the person, and includes information capable of being used for identification by being easily combined with other pieces of information even when it is impossible to identify a particular person using only the corresponding information.

The patient information irrelevant to the personal identification information may include a gender, age, height, weight, and the like of a patient.

The patient diagnosis information may include a diagnosed part, a diagnosis result, and the like.

Accordingly, the at least some pieces of the ultrasonic diagnosis image information may include at least one of the diagnosis environment information, the patient information, and the diagnosis information except the personal identification information.

Eventually, the text converter 23 may convert at least one piece among the diagnosis environment information, the patient information, and the patient diagnosis information of the ultrasonic diagnosis image information, except the personal identification information, into text data or may transmit information as it is, which already has a text data format, to the hash tag generator 24 as necessary. Although a violation of regulation of personal data protection act has to be separately discussed, the personal identification information, as necessary, may also be transmitted to the hash tag generator 24 through the text converter 23.

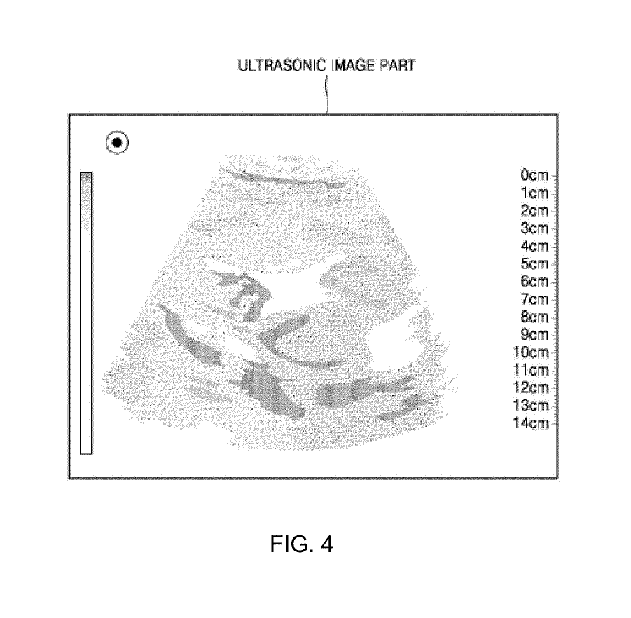

The hash tag generator 24 generates a hash tag for each piece of the received information which has been converted into text data. An example of the hash tag generated by the hash tag generator 24 is shown in FIG. 5.

Referring to FIG. 5, diagnosis environment information may include diagnosis equipment, a diagnosis position, and a diagnosis time. A hash tag of the diagnosis equipment is #Sonon300C and indicates that a type of the ultrasonic diagnosis apparatus 10 which is the diagnosis equipment is Sonon300C. A hash tag of the diagnosis position is #Korea and indicates that a position at which an ultrasonic diagnosis is performed is Korea. A hash tag of the diagnosis time is #11.26.2014 and indicates that an ultrasonic diagnosis is performed on Nov. 26, 2014.

The patient information may include a gender, age, height, and weight. A hash tag of the gender is #female and indicates that the gender of a patient is female. A hash tag of the age is #A35 and indicates that the patient is 35 years old. A hash tag of the height is #H170 and indicates that the patient is 170 cm in height. A hash tag of the weight is #W60 and indicates that the patient is 60 Kg in weight. Since it is difficult to distinguish the age, height, and weight of the patient information when only numbers of the age, height, and weight are displayed, hash tags may be generated including classification letters such as A, H, and W.

Patient diagnosis information may include a diagnosed part, a diagnosis field, and a diagnosis result.

A hash tag of the diagnosed part is #liver and indicates that the diagnosed part is a liver. A hash tag of the diagnosis field is #OB and indicates that the diagnosis field is obstetrics (OB). A hash tag of the diagnosis result is #PlacentaPrevia and indicates that the diagnosis result is placenta previa.

A hash tag may be a format of meta data which allows a particular keyword to be easily searched for on the Internet service such as social network service (SNS) and the like. For example, it is necessary to enter #BrazilWorldCupSoccer when “Brazil World Cup Soccer” is to be searched for on the Internet service such as SNS and the like, and it is necessary to enter #WorldCup when to search for “World Cup.”

Likewise, in the embodiment of the present invention, when a user intends to search for ultrasonic images related to a liver on the Internet service such as SNS and the like, it is necessary to enter #liver.

The uploader 26 includes a web Application Programming Interface (WEB API) 25 and uploads the ultrasonic image to be uploaded and the hash tag together on the Internet service such as SNS and the like.

Meanwhile, in the embodiment of FIGS. 1A and 2, it is shown that an ultrasonic diagnosis image is uploaded by the ultrasonic diagnosis apparatus 10 and the uploading terminal 20.

Unlike this, as shown in FIG. 1B, an ultrasonic diagnosis apparatus 15 may be embodied to perform both an ultrasonic diagnosis function and a function of uploading an ultrasonic diagnosis image generated according to an ultrasonic diagnosis on the Internet service.

That is, the ultrasonic diagnosis apparatus 15 may include an ultrasonic probe (not shown) which transmits an ultrasonic signal to a subject to be inspected and generates an ultrasonic diagnosis image signal by receiving and converting an ultrasonic signal (echo signal) reflected by the subject, a diagnosis image generator (not shown) which generates an ultrasonic diagnosis image using the ultrasonic diagnosis image signal obtained through the ultrasonic probe, an ultrasonic image generator which generates an ultrasonic image to be uploaded, by extracting an ultrasonic image part from the ultrasonic diagnosis image generated by the diagnosis image generator, a text converter which converts at least some pieces of ultrasonic diagnosis image information corresponding to the ultrasonic diagnosis image, into text data, a hash tag generator which generates a hash tag for each piece of information converted into the text data, and an uploader which uploads the ultrasonic image to be uploaded and the hash tag on the Internet service.

Here, the above description may be applied to descriptions of the ultrasonic image generator, the text converter, the hash tag generator, and the uploader, which are included in the ultrasonic diagnosis apparatus 15.

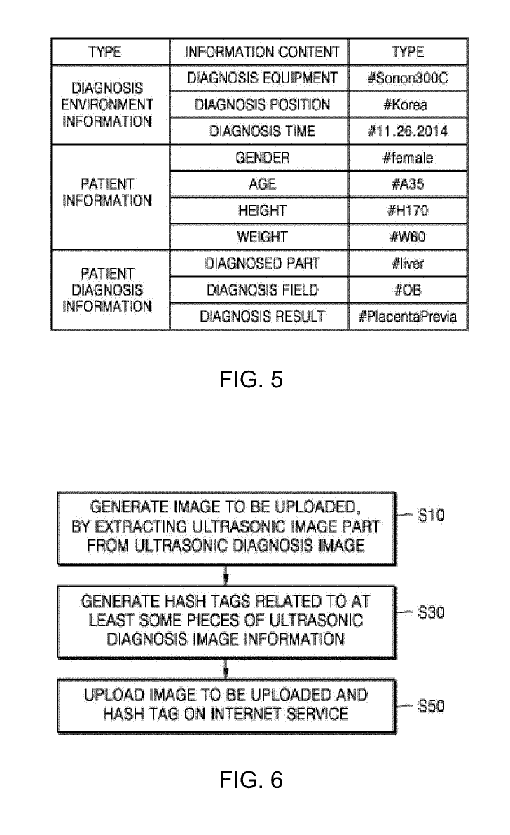

FIG. 6 is a flowchart illustrating an ultrasound diagnosis image uploading method according to one embodiment of the present invention.

Although the ultrasonic diagnosis image uploading method shown in FIG. 6 may be performed by the uploading terminal 20 shown in FIGS. 1A and 2 but is not limited to the configuration of the uploading terminal 20 shown in FIGS. 1A and 2. That is, the method may be performed by the ultrasonic diagnosis apparatus 15 which includes the configuration of the uploading terminal 20.

Hereinafter, a process of performing the ultrasonic diagnosis image uploading method according to the embodiment of the present invention will be described with reference to the configuration of the uploading terminal 20 shown in FIGS. 1A and 2. Accordingly, content described in relation to the uploading terminal 20, even omitted hereinafter, may be applied to the ultrasonic diagnosis image uploading method according to the embodiment of the present invention.

The ultrasonic image generator 22 generates an ultrasonic image to be uploaded, by extracting an ultrasonic image part from an ultrasonic diagnosis image (10).

The text converter 23 converts at least some pieces of ultrasonic diagnosis image information corresponding to the ultrasonic diagnosis image into text data, and the hash tag generator 24 generates a hash tag for each piece of information converted into a text (30).

Here, the generating, by the ultrasonic image generator 22, the ultrasonic image to be uploaded and the generating, by the text converter 23 and the hash tag generator 24, the hash tag may be performed at the same time. Otherwise, the ultrasonic image to be uploaded is generated first, and then, the hash tag is generated. Otherwise, the hash tag is generated first, and then, the ultrasonic image is generated.

After the ultrasonic image and the hash tag are generated as described above, the uploader 26 uploads the ultrasonic image to be uploaded and the hash tag together on the Internet service (50).

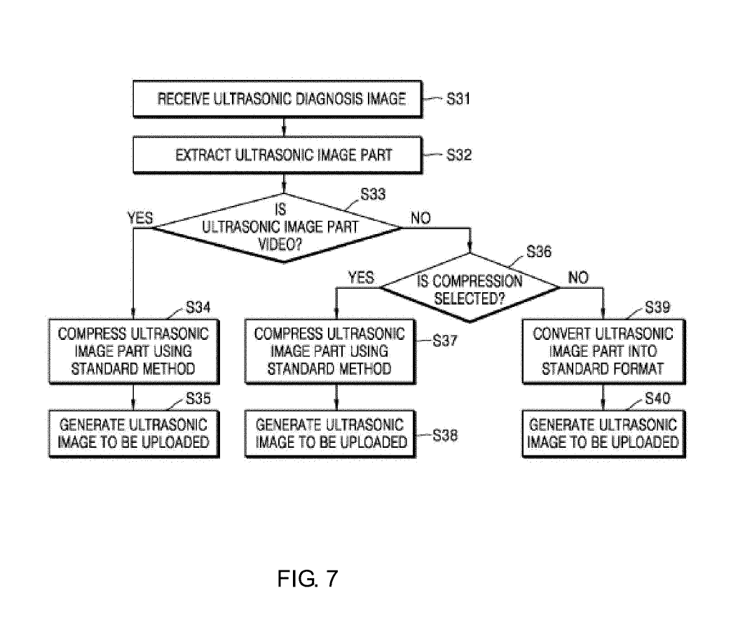

FIG. 7 is a flowchart illustrating a method of generating an ultrasound image to be uploaded according to one embodiment of the present invention.

The method of generating an ultrasonic image to be uploaded, shown in FIG. 7, may be performed by the ultrasonic image generator 22 shown in FIG. 2. Accordingly, content described in relation to the ultrasonic image generator 22, even omitted hereinafter, may be applied to the method of generating an ultrasonic image to be uploaded according to the embodiment of the present invention.

The ultrasonic image generator 22 receives an ultrasonic diagnosis image obtained by the ultrasonic diagnosis apparatus 10 (31).

Afterward, the ultrasonic image generator 22 extracts an ultrasonic image part from the ultrasonic diagnosis image (32).

The ultrasonic image generator 22 checks whether the extracted ultrasonic image part is a video or a still image (33).

When the extracted ultrasonic image part is a video, the ultrasonic image generator 22 generates an ultrasonic image to be uploaded, by compressing the extracted ultrasonic image part using a standard video coding method (34 and 35).

Meanwhile, when the extracted ultrasonic image part is a still image, the ultrasonic image generator 22 checks whether compression is selected (36).

When compression is selected, the ultrasonic image generator 22 generates the ultrasonic image to be uploaded, by compressing the ultrasonic image part which is a still image, using a standard still image coding method. Here, a format of the ultrasonic image to be uploaded, which is generated by compressing the ultrasonic image part which is a still image, using the standard still image coding method may be JPEG.

When compression is not selected, the ultrasonic image generator 22 generates an ultrasonic image to be uploaded, which has a standard incompressible still image format, using the ultrasonic image part which is a still image. Here, the standard incompressible still image format may be PNG.

Since a hash tag corresponding to an ultrasonic image part may be automatically generated and may be uploaded on the Internet service through the above operations, a large number of ultrasonic images of a patient may be collected in one space on the Internet and may be variously classified or collected through hash tags automatically generated for each of the large number of ultrasonic images so as to be utilized for education, seminar, or the like of medical personnel.

According to the embodiments of the present invention, a method and a system for uploading an ultrasonic diagnosis image may automatically extract and upload an ultrasonic diagnosis image instead of manually capturing and uploading, by a user, an ultrasonic diagnosis image on the Internet service so as to provide an advantage of uploading a large number of high-definition ultrasonic diagnosis images. Accordingly, joint treatment among doctors, education, first aid, and the like may be supported using a variety of more accurate ultrasonic diagnosis images.

Also, since it is possible to automatically convert and compress an ultrasonic diagnosis image into an adequate format for the Internet service depending on whether the ultrasonic diagnosis image is a still image or a video, there is an advantage of providing a high-definition ultrasonic diagnosis image to many people for public interest.

Also, since it is possible to automatically select and upload a variety of pieces of information which describe an ultrasonic diagnosis image and to automatically convert the information into a unified hash tag format to upload, the ultrasonic diagnosis image may be searched for on all types of the Internet service.

Ultrasonic diagnosis information corresponding to an ultrasonic diagnosis image is converted into a hash tag and uploaded with an ultrasonic image part of the ultrasonic diagnosis image on the Internet service such that personal information for identifying a patient may be prevented from being exposed while being shared on the Internet service such as SNS and the like.

Also, a user may easily search for an ultrasonic diagnosis image on the Internet such as SNS and the like by using a hash tag.

Due the above-described a variety of advantages, the method and system for uploading an ultrasonic diagnosis image according to the embodiments of the present invention may activate processing a variety of types of accurate ultrasonic diagnosis images related to diseases or body parts to be collected on the Internet service as big data such that the big data may activate joint treatment and education in a medical team and may be helpful for emergency crews to give first aid.

While the embodiments of the present invention have been described above, it should be understood by one of ordinary skill in the art that modifications may be made without departing from the essential features of the present invention. Therefore, the disclosed embodiments should be considered not in a limitative viewpoint but in a descriptive viewpoint. Accordingly, it should be understood that the scope of the present invention is not limited to the above-described embodiments and includes a variety of modifications within the range of the content defined in the claims and equivalents thereof.

1. A method of uploading an ultrasonic diagnosis image, comprising:
generating an ultrasonic image to be uploaded, by extracting an ultrasonic image part from an ultrasonic diagnosis image;
generating a hash tag by converting at least some pieces of ultrasonic diagnosis image information corresponding to the ultrasonic diagnosis image, into text data; and
uploading the ultrasonic image to be uploaded and the hash tag together on the Internet service.

2. The method of claim 1, wherein the generating of the ultrasonic image to be uploaded comprises, when the extracted ultrasonic image part is a video, generating an ultrasonic image to be uploaded, by compressing the extracted ultrasonic image part using a standard video coding method.

3. The method of claim 1, wherein the generating of the ultrasonic image to be uploaded comprises, when the extracted ultrasonic image part is a still image, generating an ultrasonic image to be uploaded, by compressing the extracted ultrasonic image part using a standard still image coding method or generating an ultrasonic image to be uploaded, which has a standard incompressible still image format.

4. The method of claim 1, wherein the at least some pieces of information comprises at least one of diagnosis environment information, patient information, and patient diagnosis information.

5. The method of claim 4, wherein the diagnosis environment information comprises at least one of diagnosis equipment, a diagnosis position, and a diagnosis time.

6. The method of claim 4, wherein the patient information comprises at least one of a gender, age, height, and weight.

7. The method of claim 4, wherein the patient diagnosis information comprises at least one of a diagnosed part, a diagnosis field, and a diagnosis result.

8. An ultrasonic diagnosis image uploading system comprising:
an ultrasonic diagnosis apparatus; and
an uploading terminal which comprises an ultrasonic image generator which generates an ultrasonic image to be uploaded, by extracting an ultrasonic image part from an ultrasonic diagnosis image obtained through the ultrasonic diagnosis apparatus, a text converter which converts at least some pieces of ultrasonic diagnosis image information corresponding to the ultrasonic diagnosis image, into text data, a tag generator which generates a hash tag for each piece of information converted into the text data, and an uploader which uploads the ultrasonic image to be uploaded and the hash tag together on the Internet service.

9. The system of claim 8, wherein the at least some pieces of information comprises at least one of diagnosis environment information, patient information, and patient diagnosis information.

10. An ultrasonic diagnosis apparatus comprising:
an ultrasonic probe which generates an ultrasonic diagnosis image signal by converting an ultrasonic signal reflected by a subject to be inspected;
a diagnosis image generator which generates an ultrasonic diagnosis image using the ultrasonic diagnosis image signal obtained through the ultrasonic probe;
an ultrasonic image generator which generates an ultrasonic image to be uploaded, by extracting an ultrasonic image part from the ultrasonic diagnosis image generated by the diagnosis image generator;
a text converter which converts at least some pieces of ultrasonic diagnosis image information corresponding to the ultrasonic diagnosis image, into text data;
a hash tag generator which generates a hash tag for each piece of information converted into the text data; and
an uploader which uploads the ultrasonic image to be uploaded and the hash tag on the Internet service.

11. (canceled)
